(19) United States
(12) Patent Application Publication
(10) Pub. No.: US 2015/0272766 A1
(43) Pub. Date: Oct. 1, 2015

(54) KNEE BRACE AND STRAP FOR USE THEREWITH

(71) Applicant: Tony DARUGAR, Eatonton, GA (US)

(72) Inventor: Tony DARUGAR, Eatonton, GA (US)

(21) Appl. No.: 14/670,894

(22) Filed: Mar. 27, 2015

Related U.S. Application Data
(60) Provisional application No. 61/971,839, filed on Mar. 28, 2014.

(51) Int. Cl. A61F 5/01

(52) U.S. Cl. CPC A61F 5/0123

(57) ABSTRACT

A knee brace including a pair of longitudinal supports, a plurality of primary straps, and a plurality of secondary straps overlaying the primary straps. The plurality of primary straps each include an inner layer and an outer layer. Optionally, the primary strap may be used as a musculoskeletal support.

### BACKGROUND

Knee braces and joint support straps are commonly known for supporting the knee or other joints such as the shoulder, elbow, foot, hand, arm, etc. Commonly, braces and support straps can be difficult to apply, do not allow for adequate support as they wear over time, and often move out of place or migrate out of position. Furthermore, braces and supports have been known to be uncomfortable and retain moisture.

Thus, it can be seen that needs exist for a knee brace and strap for use therewith. It is to the provision of a knee brace and strap for use therewith meeting these and other needs that the present invention is primarily directed.

### SUMMARY OF THE INVENTION

In a first example form, the present invention relates to a knee brace including a pair of longitudinal supports, a plurality of primary straps, and a plurality of secondary straps overlaying the primary straps. The primary straps each include an inner layer and an outer layer affixed to one another. In one optional form, the inner layer includes a polymer material and the outer layer includes an elastic fabric. Preferably, the polymer material is in the form of a fabric comprising a mesh-like grid pattern and the elastic fabric is in the form of an elastic bandage.

Optionally, the polymer material includes a rubberized material for frictionally engaging a wearer's skin or clothing. In one form, at least a portion of each of the primary straps includes hook and loop material. The hook and loop material removably couples the primary straps to the longitudinal supports.

In one optional form, at least four primary straps and at least four secondary straps are provided for removably securing the knee brace to a wearer's leg.

In second example form, the present invention relates to a knee brace including a pair of longitudinal supports, a plurality of primary straps, and a plurality of secondary straps overlaying the primary straps. In one form, the primary straps include an inner layer and an outer layer that are generally affixed to one another. The inner layer includes a synthetic material and the outer layer includes an elastic fabric. In some forms, the synthetic material is in the form of a fabric comprising a mesh-like grid pattern and the elastic fabric is in the form of an elastic bandage.

Optionally, the synthetic material includes a rubberized material for frictionally engaging a wearer's skin or clothing.

In one optional form, at least a portion of each of the plurality of primary straps includes hook and loop material. In some forms, hook and loop material removably couples the primary straps to the longitudinal supports.

In a third example form, the present invention relates to a post-operative orthopedic knee brace including a pair of longitudinal supports and a plurality of primary straps. Each of the primary straps includes an inner layer and an outer layer. The inner layer includes a soft material with good skin-to-material friction and the outer layer includes an elastic fabric. In one form, the soft material is in the form of a fabric having a mesh-like grid pattern and the elastic fabric is in the form of an elastic bandage. Optionally, the soft material includes a rubberized material for frictionally engaging a wearer's skin or clothing. Optionally, the post-operative orthopedic knee brace includes a plurality of secondary straps overlaying the primary straps.

In another example form, the present invention relates to a musculoskeletal support including an outer layer and an inner layer. The outer layer has a length, a width, a first end and a second end, and is formed from an elastic material. The inner layer has a length, a width, a first end and a second end, and is formed from a synthetic material. In example forms, the outer layer and the inner layer are affixed to one another and the outer layer has an unstretched length that is less than the length of the inner layer. Optionally, the first and second ends of the strap include hook and loop material for releasable engagement therewith.

### BRIEF DESCRIPTION OF THE DRAWING FIGURES

FIG. 1 is an outside plan view of a knee brace according to an example embodiment of the present invention

FIG. 2 is an inside plan view of the knee brace shown in FIG. 1.

FIG. 3A-B are top schematic views of portions of a primary strap shown in FIGS. 1-2.

FIG. 4 is an inside plan view of a joint support strap according to another example embodiment of the present invention.

FIG. 5 is an outside view of the joint support strap shown in FIG. 4.

FIG. 6A-B are top, schematic views of portions of a secondary strap shown in FIGS. 1-2.

FIG. 7A-B show a child without and with the primary straps of FIGS. 4-5 spiraled around the legs thereof and applying a torque thereto according to another example embodiment of the present invention.

### DESCRIPTION OF EXAMPLE EMBODIMENTS

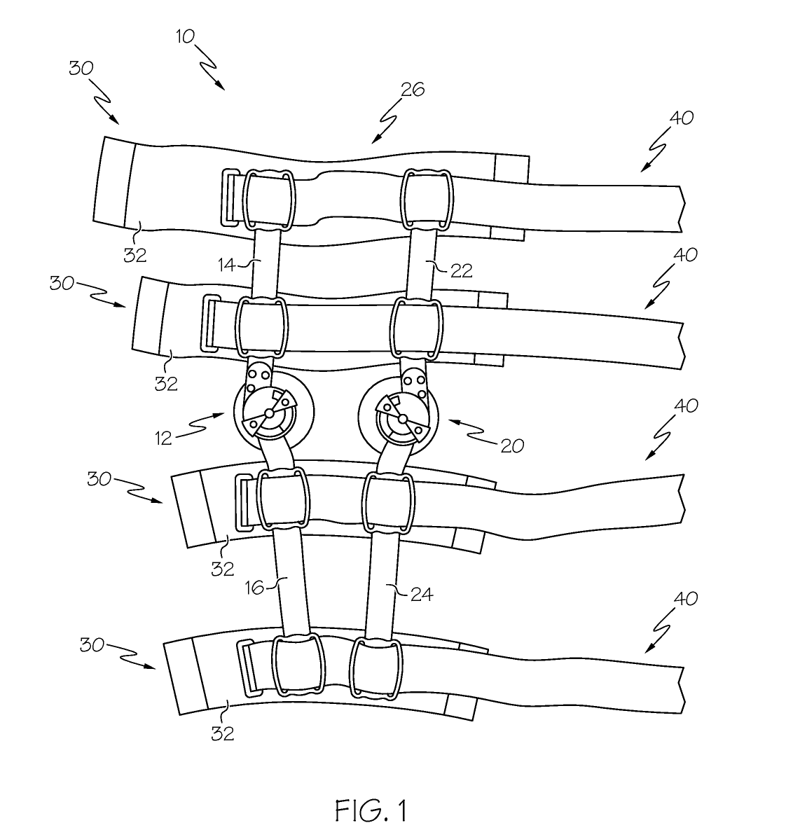

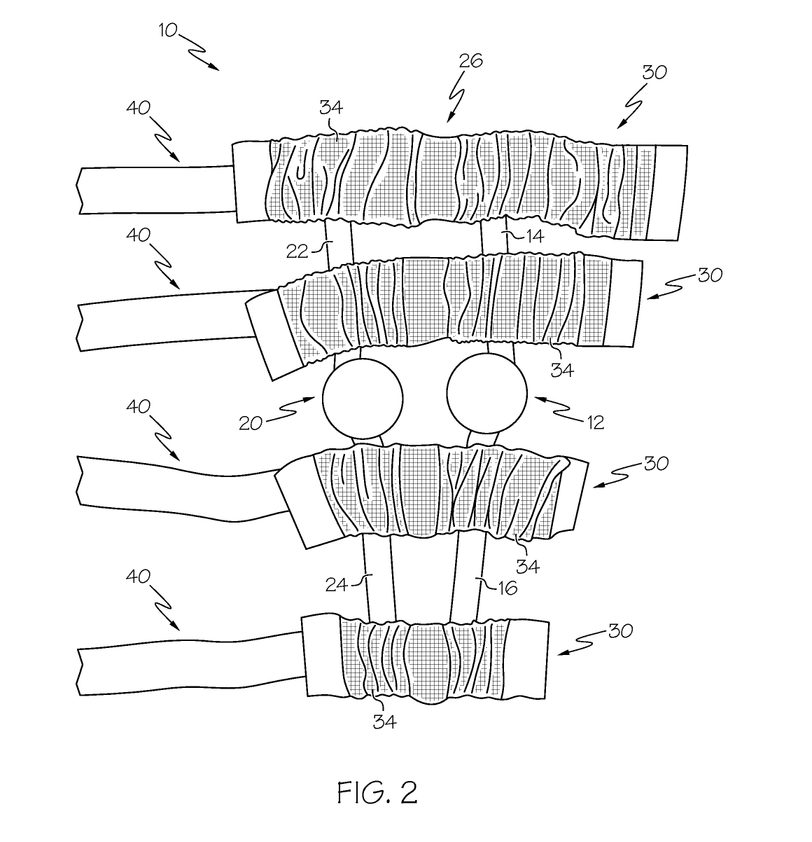

Referring now to the drawing figures, in which like numerals refer to like elements throughout the several views, FIGS. 1-2 show a musculoskeletal support or orthopedic knee brace 10 according to an example embodiment of the present invention. Generally, the knee brace 10 is to be worn on the leg of a user to stabilize and support the user's knee, or areas thereby, which is generally referred to a leg or knee portion of the human musculoskeletal support system, while being comfortable, secure, and breathable. Preferably, the knee brace 10 is generally universal such that it can be worn on either the left or right leg and can accommodate legs of different sizes. Optionally, the knee brace 10 (and its components, as will be described below) can be sized to fit a leg of a particular size or a particular range of sizes.

Generally, the knee brace 10 comprises a pair of longitudinal support assemblies 12, 20 and a strapping assembly 26 including a plurality of primary straps 30 and a plurality of secondary straps 40. In one example form, the strapping assembly 26 comprises four primary straps 30 and four secondary straps 40. Preferably, the longitudinal support assemblies 12, 20 are not limited to any one configuration. Any number of longitudinal support assemblies, which are well known in the art, can be equally applicable to the knee brace 10. Furthermore, braces, connecting members or assemblies used with prosthetics or coupling members, straps, or other engagement members thereof, may be provided with at least the primary straps as described herein.

In one example embodiment, the longitudinal support assemblies 12, 20 generally comprise upper stays 14, 22 and lower stays 16, 24, which are generally pivotally mounted to each other, respectively. In typical embodiments, the longitudinal support assemblies 12, 20 are configured to be placed adjacent the inner and outer sides of the user's leg such that the pivots (generally centrally located between the upper and lower stays 14, 22, 16, 24) are generally aligned with the knee, thereby providing support to the knee. As depicted in FIGS. 1-2, for attaching the brace 10 to the user's left leg, the longitudinal support assembly 20 is positioned adjacent the outer side of the leg and the longitudinal support assembly 12 is positioned adjacent the inner side of the leg. And, for attaching the brace 10 to the user's right leg, the longitudinal support assembly 20 is positioned adjacent the inner side of the leg and the longitudinal support assembly 12 is positioned adjacent the outer side of the leg.

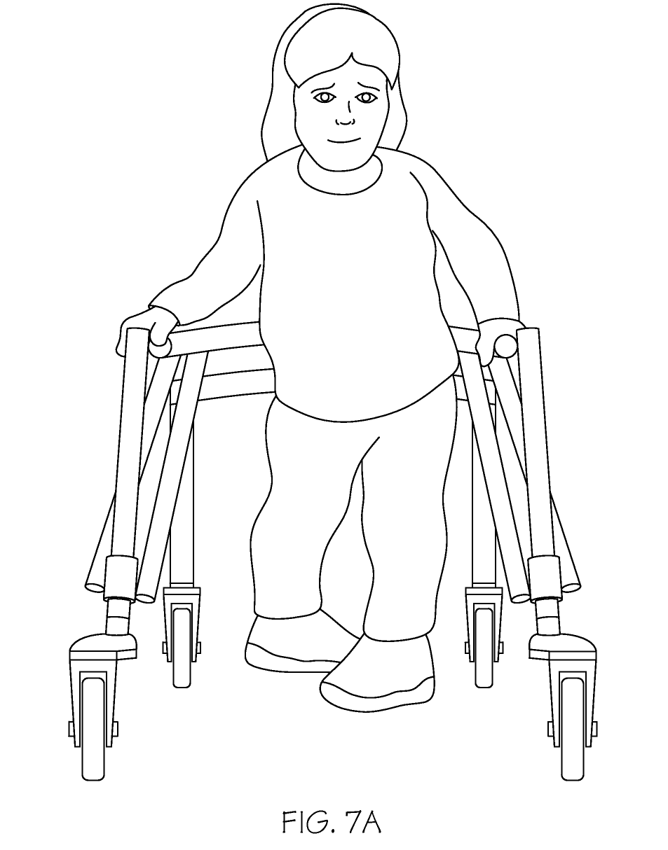

The plurality of primary straps 30 and secondary straps 40 are generally spaced along the longitudinal support assemblies 12, 20 and are generally oriented and positioned generally transverse to the extension of the stays 14, 16, 22, 24. Generally, the plurality of primary straps 30 are affixed to a first side of the stays and the plurality of secondary straps 40 are affixed to a second side of the stays. In one example form, a fastener, such as hook and loop material, is provided for securing the plurality of primary straps to the stays, and a plurality of buckles 52 (see FIGS. 6A-B) are provided for securing the plurality of secondary straps 40 to the stays. Typically, the buckles 52 are affixed to the stays in a conventional fashion and the secondary strap 40 is looped through the buckle (see FIG. 6B). Optionally, other fasteners, for example, snaps, clips, stitching, adhesives, inter-engaging members, coupling elements or other fasteners, may be provided to permanently or releasably secure the primary straps 30 and the secondary straps 40 to the stays.

Preferably, the strapping assembly 26 is configured such that the secondary straps 40 generally overlay the primary straps 30. Thus, in example embodiments, the primary straps 30 are generally wrapped around the user's leg and secured thereto, and then the secondary straps 40 are wrapped around the primary straps 30 and secured thereto, thereby providing a two-piece overlaid strapping system.

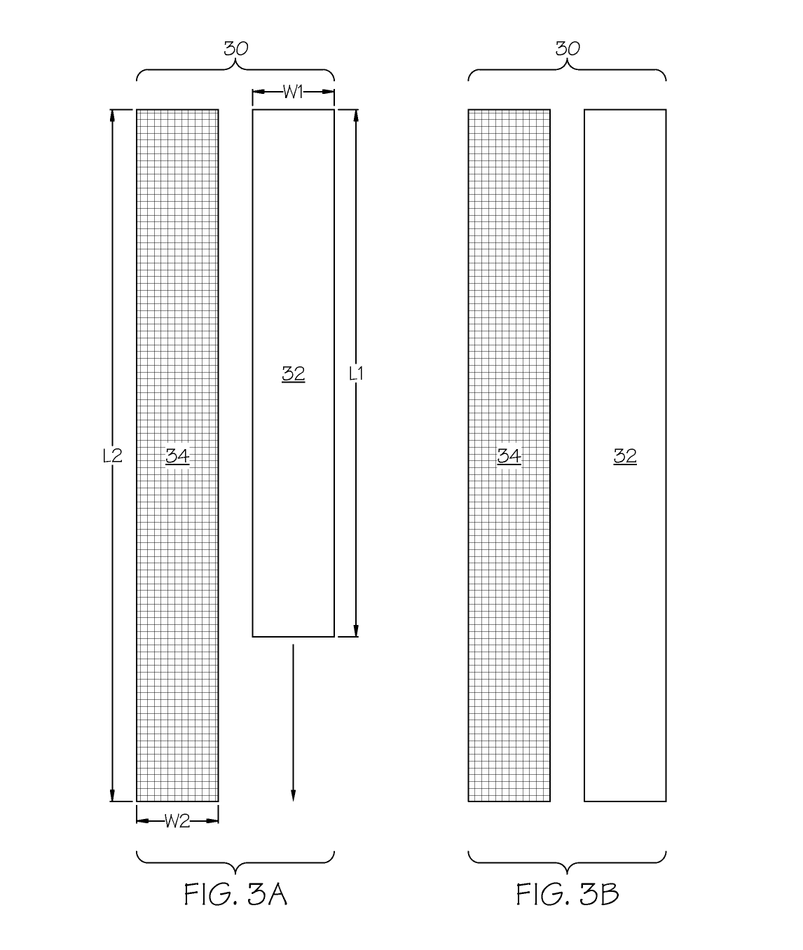

FIGS. 3A-B show components of the primary strap 30 including an outer member 32 and an inner member 34, which are generally affixed to one another to form the primary strap 30. In one example form, the outer member 32 is generally formed from an elastic fabric and the inner member 34 is generally formed from a synthetic material, such as a polyvinyl chloride (PVC) fabric. Other polymer materials can be used as well. Preferably, the material is a soft material with good skin-to-material friction. In one form, the elastic fabric is in the form of an elastic bandage, for example an Ace™ brand bandage, and the PVC fabric is in the form of a soft rubberized PVC material, for example Arm & Hammer™ antimicrobial lining material. In some example forms, the PVC fabric is soft, mesh-like, antimicrobial, breathable, and has high bio-compatibility and odor control. Optionally, other fabrics or textiles such as nylons, cottons, polyesters, acrylics, rubbers, plastics, other materials or combinations thereof can be provided to construct the outer and/or inner members 32, 34.

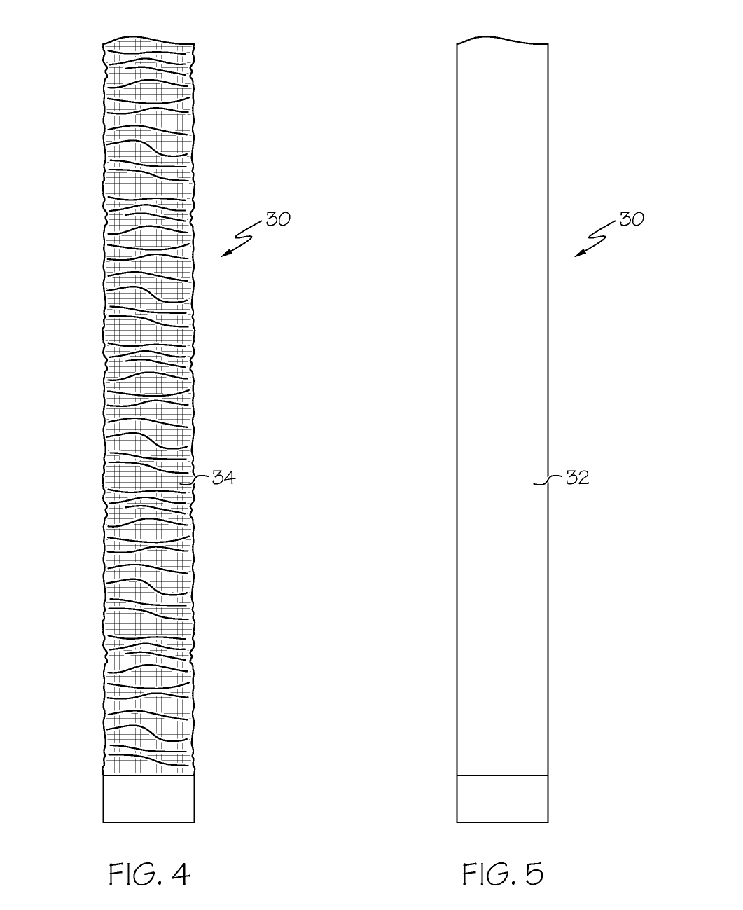

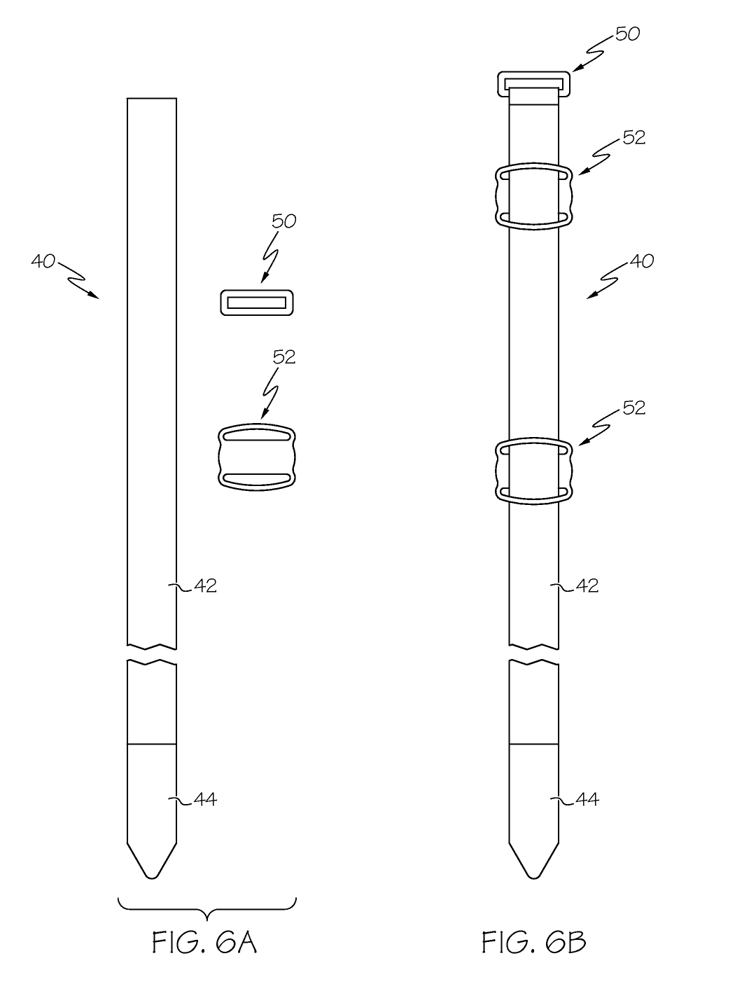

When constructing the primary strap 30, the outer and inner members 32, 34 preferably are of different lengths such that the inner member 34 is somewhat ruffled after being affixed to the outer member 32. For example, the outer member 32 preferably has a length L1 and a width W1, while the inner member 34 has a length L2 and a width W2. Preferably, when the outer member 32 is in its relaxed, unstretched state, the length L1 is about 25% less than the length L2 (see FIG. 3A). And, when the outer member 32 is expanded to a stretched state, the length L1 is generally equal to the length L2 (see FIG. 3B). Thus, when affixing the outer and inner members 32, 34 together, the outer member 32 preferably remains in a stretched state while the two are affixed together. Optionally, the length L1 is between about 25%-65% less than the length L2. Then, when the outer member 32 retracts toward its relaxed, unstretched state, the inner member 34 comprises a plurality of wave-like ruffles along the length of the strap 30 (see FIGS. 4-5). Typically, the outer and inner members 32, 34 are affixed to one another by generally stitching or threading along the outer periphery portions thereof. Optionally, the outer and inner members 32, 34 can be affixed to one another in a variety of other ways including but not limited to adhesives, coupling elements, heat bonding, clips, snaps, or other fasteners. Preferably, the lengths L1, L2 and widths W1, W2 can be sized as desired. In one form, the lengths L1, L2 will generally vary depending on where they are positioned on the stays of the longitudinal support assemblies 12, 20, for example, to generally remain proportional to the portion of the leg that it is to be wrapped around and secured to. Optionally, the lengths L1, L2 are configured to provide a generally universal length to accommodate a wide range of leg sizes.

Preferably, the one or more coupling elements are provided near the ends of the primary strap 30 such that the straps can be wrapped and secured to the user's leg. In one example form, the ends of the primary strap 30 comprise hook and loop material, for example industrial grade Velcro®, which allows for removably securing the strap 30 around the user's leg at a desired tension. Thus, one end of the primary strap 30 generally comprises hook material and the other (opposite) end generally comprises loop material. Optionally, one end of the primary strap comprises hook material that is configured for removable engagement with at least a portion of the outer member 32, for example wherein the material of the outer member 32 is capable of releasable engagement with the hook material. Also, other engagement features, including snaps, clips, t-stay buckles, or other fasteners, may be provided to accommodate engagement of the primary strap 30 such that it can be wrapped around the user's leg and secured with a particular level of tension.

FIGS. 6A-B show the secondary strap 40 in greater detail. Generally, the secondary strap 40 comprises an elongate strap comprising a first portion 42 extending from a first end towards a second end, and a second portion 44 extending from the second end towards the first end. Typically, the first portion 42 comprises loop material and the second portion 44 comprises hook material (or vice versa). A first buckle 50 preferably is provided at the first end and a plurality of second buckles 52 are provided between the first and second ends. Typically, the second buckles 52 provide for securing the secondary strap 40 to the stays 14, 16, 22, 24, for example wherein the buckles 52 are generally mounted to the stays in a conventional fashion and the strap 40 is looped through the buckles 52. Optionally, the buckles 52 may be integrally formed with the stays. The first buckle 50 is configured to allow for the second end to be looped there through such that the second portion 44 (hook material) can be removably secured to the first portion 42 (loop material) once the strap 40 is wrapped around the primary strap 30. Optionally, the second portion 44 can comprise loop material and the first portion 42 can comprise hook material. Also, other coupling elements, such as snaps, clips, or other engagement features, may be provided.

In use, preferably the knee brace 10 is placed on a user's leg wherein the longitudinal support assemblies 12, 20 are positioned adjacent the inner and outer sides of the user's leg, and the strapping assembly 26 is then wrapped around the leg and secured thereto. Preferably, the straps 30 are configured such that the inner member 34 contacts and remains engaged with the skin of the user's leg, which allows for comfort, substantial grip, secure support, breathability, soft feel, extra cushioning, and prevents migration of the brace 10. Thus, the PVC inner member 34 preferably provides substantial gripping capability to prevent the strap 30 from moving on the leg.

In example forms, preferably the primary straps 30 are secured to the user's leg first by grasping both ends of the strap 30, wrapping the ends around the leg, and then securing the ends together, or alternatively securing one end to another portion of the strap 30. Preferably, the strap 30 provides tension adjustment by stretching the strap as desired. Thus, comfort can be achieved easily by adjustment to the tension of the strap 30. After the primary straps 30 are secured to the user's leg, the secondary straps 40 (generally overlaying the primary straps 30) are wrapped around the primary straps 30 and secured thereto, thereby providing a two-piece overlaid strapping system. Optionally, the knee brace 10 can be applied over a garment of clothing or on the outside of clothes, for example a user's pant leg, while remaining positioned as desired.

In additional example embodiments, preferably the primary strap 30 can be used as a joint support wrap or musculoskeletal support. As similarly described above with regard to wrapping and securing the primary strap to the user's leg when attached to the brace 10, the primary strap 30 can be utilized as a single strap to support individual joints, for example, the shoulder, knee, elbow, ankle/foot, and/or hand/wrist/arm. Preferably, the primary strap 30 provides in actively assisting the joint that the strap is applied with movement while the joint is weak.

### Testing

The durability of the inner member 34 was extensively tested. In one example form, it was lined over shoe soles inside shoes and walked on for about 30 weeks. The material was found to add cushion with comfort and grip on the sole of the foot. In addition, it was found that the material prevented sliding of the foot in the shoe and proved durable. Also, the material of the inner member 34 is bio-compatible with skin. It was found that it feels good on the sole of bare feet since it is soft and cushioned, while allowing a good grip on the feet. This makes it compatible with possible use on open toe shoes for extra grip, comfort, durability, and antimicrobial properties.

Another test for durability of the inner member 34 was using it as a shower mat to prevent slipping in the shower with a bath tub. The inner member 34 is thus waterproof and washable making it durable over time in the shower with a tub, which was tested for about 40 weeks. Again, the inner member 34 feels good to bare feet in the tub making it comfortable to stand on with cushion, grip, and durability.

Testing of the primary strap 30 as a knee support provided compression and prevented swelling. Also, the inner member 34 lining outer member 32 allows some stretching with the outer member 32, creating more spring-like active assistive action for knee movement with good support, comfort, grip, and compression. For example, with weak quadriceps muscles, the active assistive action of the primary strap 30 helped knee extension during walking as the weak quadriceps fatigued. Support during stair negotiation was much better with the primary strap 30 considering recovery from a quadriceps tendon repair. In addition, exercising the knee with the primary strap 30 allows much more support for safety with strengthening the tendons of the knee. The primary strap 30 does not have to be very long, which makes it easy to wrap around the knee a few times for very good support. Also, the primary strap 30 allowed some spring-action actively assisting the knee since the inner member 34 allows some stretch along with the outer member 32. In one test, the primary strap 30 was used on a subject for support with an arthritic knee. The subject stated that the primary strap 30 allowed much support making a day of walking much better and stable with comfort.

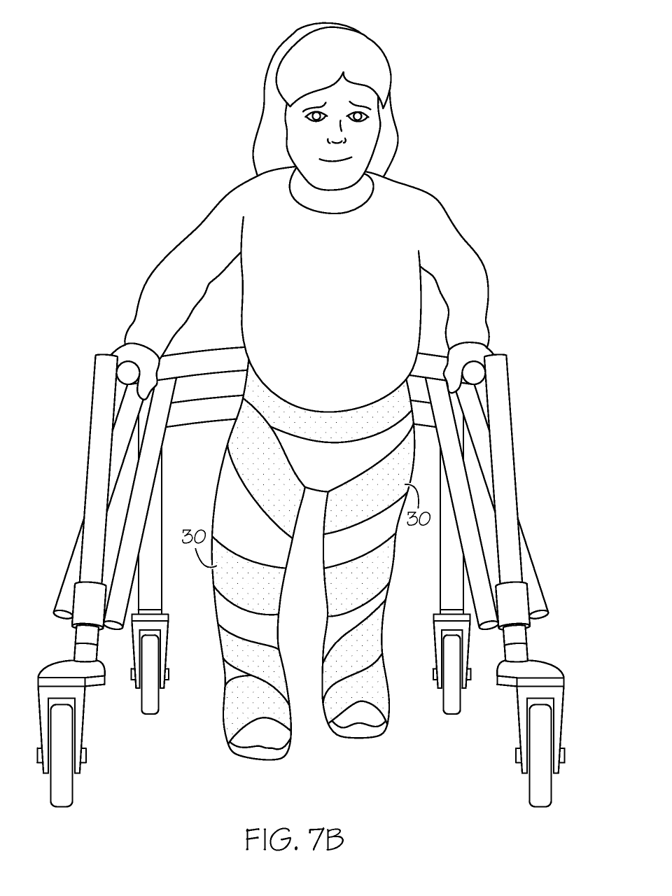

Furthermore, the brace and/or straps of the present invention preferably provides musculoskeletal support with tone and positioning. For example, the straps have proved to help position and stabilize legs, hips, and shoulders that are not in the right anatomical position to allow better functioning with basic activities of daily living. FIGS. 7A-B show an actual before and after result of a spina bifida juvenile patient aided by the primary strap 30 of the present invention. As shown in FIG. 7A, a child with spina bifida may have their feet generally inwardly rotated, for example, such that when the child walks their feet collide with each other. In example forms, at least one primary strap 30 can be wrapped around the foot and then spiraled up the leg to help outwardly rotate the leg or provide torque thereto, for example to allow proper positioning of the feet for more normal walking. As shown in FIG. 7B, the child brace comprises a primary strap 30 for each leg, wherein the strap is spiraled up the child's legs, starting at the feet, and spiraled coming from the interior of each foot outwardly around the leg, thereby applying a torque thereto such that the feet are forced outwardly to a more normal forward-facing, spaced-apart orientation. Tension is provided on the straps such that the child's legs are positioned in a more normal orientation with the feet thereof generally spaced apart and facing forwardly. Preferably, the strap may be any desired length and width. In one example form, the strap 30 is between about 2-14 feet in length and generally between about 3-8 inches in width. In example forms, the strap comprises hook (on loop) material at each of the ends, and at least one side of the strap either comprises loop (on hook) material or is generally capable of removable engagement with hook material. Optionally, other fasteners, engagement features, adjusting members, loops, or other straps, etc. may be used as desired. As such, the primary strap 30 corrects the legs and hips for a more normal anatomical position and prevents joint problems. Another benefit is that the legs and trunk can be strengthened with various exercises while the straps hold the legs and trunk in its more normal anatomical position.

According to another example form, at least one primary strap can be utilized to help tone and position the legs, shoulders, and trunk of person who has had a stroke. In one case, a person had their right leg and foot externally rotated and their right hip was rotated back. Preferably, the at least one primary strap can be made long enough to accommodate stretching and positioning the legs and hips in a more normal anatomical position to allow better functioning when walking.

In addition, if the right shoulder is affected by a stroke it may dip down to the right. In this case, the arm and shoulder can be lifted, supported, and pulled back up into a more normal anatomical position with such a strap. As such, the at least one primary strap can be used for toning and positioning one or more portions of a person having many deformities with the body along with joint support and bracing for the body to prevent sliding of the brace.

It is to be understood that this invention is not limited to the specific devices, methods, conditions, or parameters described and/or shown herein, and that the terminology used herein is for the purpose of describing particular embodiments by way of example only. Thus, the terminology is intended to be broadly construed and is not intended to be limiting of the claimed invention. For example, as used in the specification including the appended claims, the singular forms “a,” “an,” and “one” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein.

While the invention has been shown and described in exemplary forms, it will be apparent to those skilled in the art that many modifications, additions, and deletions can be made therein without departing from the spirit and scope of the invention as defined by the following claims.

What is claimed is:

1. A postoperative orthopedic knee brace comprising:
a pair of longitudinal supports; and
a plurality of primary straps, each of the plurality of straps comprising an inner layer and an outer layer affixed to one another; and
wherein the inner layer includes a soft material with a good skin-to-material friction for keeping the brace in place on the skin of a user.

2. The postoperative orthopedic knee brace of claim 1, further comprising a plurality of secondary straps overlaying the primary straps.

3. The postoperative orthopedic knee brace of claim 1, wherein the inner layer comprises a polymer material and the outer layer comprises an elastic fabric.

4. The postoperative orthopedic knee brace of claim 1, wherein the polymer material is in the form of a fabric comprising a mesh-like grid pattern.

5. The postoperative orthopedic knee brace of claim 1, wherein the elastic fabric is in the form of an elastic bandage.

6. The postoperative orthopedic knee brace of claim 2, wherein at least four primary straps and at least four secondary straps are provided for removably securing the knee brace to a wearer's leg

7. The postoperative orthopedic knee brace of claim 1, wherein the inner layer further comprises a rubberized material for frictionally engaging a wearer's skin or clothes.

8. A musculoskeletal support comprising:
an outer layer comprising a length, a width, a first end and a second end, the outer layer comprising an elastic material; and
an inner layer comprising a length, a width, a first end and a second end, the inner layer formed from a polymer material,
wherein the outer layer and the inner layer are affixed to one another, and wherein the outer layer comprises an unstretched length that is less than the length of the inner layer.

9. The musculoskeletal support of claim 8, wherein the inner layer is placed against a wearer's pants, clothes, or directly in contact with their skin.

10. The musculoskeletal support of claim 8, wherein the support can be spiraled around a leg of a wearer such that a torque can be applied thereto.

11. The musculoskeletal support of claim 10, wherein the torque being applied thereto generally causes the feet thereof to torque outwardly and away from each other.

12. The musculoskeletal support of claim 8, wherein the support can be utilized to tone and position one or more legs, arms and/or the hip of a wearer.

13. A knee brace comprising:
a pair of longitudinal supports;
a plurality of primary straps; and
a plurality of secondary straps overlaying the primary straps.

14. The knee brace of claim 13, wherein the plurality of primary straps comprise an inner layer and an outer layer generally affixed to one another.

15. The knee brace of claim 14, wherein the inner layer comprises a polymer material and the outer layer comprises an elastic fabric.

16. The knee brace of claim 15, wherein the polymer material is in the form of a fabric comprising a mesh-like grid pattern.

17. The knee brace of claim 15, wherein the elastic fabric is in the form of an elastic bandage.

18. The knee brace of claim 16, further comprising a rubberized material for frictionally engaging a wearer's skin or clothing.

19. The knee brace of claim 13, wherein at least four primary straps and at least four secondary straps are provided for removably securing the knee brace to a wearer's leg.
